(19) United States
(12) Patent Application Publication
(10) Pub. No.: US 2022/0127671 A1
(43) Pub. Date: Apr. 28, 2022

(54) METHODS FOR IMPROVING HUMAN EGG QUALITY USING MICRORNAs

(71) Applicant: THE JOHNS HOPKINS UNIVERSITY, Baltimore, MD (US)

(72) Inventors: Ping Xia, Baltimore, MD (US); Samarjit Das, Baltimore, MD (US); Bhuchitra Singh, Baltimore, MD (US)

(21) Appl. No.: 17/506,327

(22) Filed: Oct. 20, 2021

Related U.S. Application Data
(60) Provisional application No. 63/106,717, filed on Oct. 28, 2020.

(51) Int. Cl. C12Q 1/6876; C12N 5/075

(52) U.S. Cl. CPC C12Q 1/6876; C12N 2501/65; C12Q 2600/158; C12N 5/0609

(57) ABSTRACT

The disclosure is directed to methods for improving the quality of oocytes for assisted reproductive technology procedures (ARTs), such as oocyte cryopreservation and in vitro fertilization. The method involves culturing an oocyte in which endogenous expression of miRNAs miR-20b, miR-199, and miR-363 is absent or reduced in the presence of exogenous miR-20b, miR-199, and miR-363. The disclosure also provides methods for selecting high-quality oocytes by assessing expression of the miRNAs miR-20b, miR-199, and miR-363 in a cumulus cell surrounding an oocyte isolated from a subject, and methods for performing an ART procedure using a selected high-quality oocyte.

### REFERENCE TO RELATED APPLICATIONS

This application claims priority to U.S. provisional patent application Ser. No. 63/106,717, filed Oct. 28, 2021, which is incorporated herein by reference in its entirety.

### BACKGROUND

Approximately 39.48 million (10.5%) of women experience infertility globally. In the U.S. alone, about 10% of women (6.1 million) ages 15-44 experience difficulty getting pregnant according to the Centers for Disease Control and Prevention (CDC). There is an acute limitation in objective analysis of the quality of human eggs (oocytes) before choosing an oocyte to proceed with Assisted Reproductive Technology (ART) procedures, such as in vitro fertilization (IVF) and elective human egg freezing for fertility preservation (cryopreservation). Oocyte quality is typically determined by subjective morphological analysis, which potentially contributes to the selection of low yield oocytes for ART procedures and low success rates.

ART procedures are high cost and often not covered by insurance policies. The low success rates put an immense pressure on the individual, family, and reproductive endocrinologists/fertility experts. For younger women interested in oocyte freezing for fertility preservation, there is no avenue to store the oocytes that ensures the best yield. The long term storage of oocytes also comes at a large cost, along with psychological and emotional burden. Furthermore, the majority of patients seeking IVF treatment typically undergo more than one cycle due to its high failure rate (e.g., embryo failed to implant, not viable, etc.). For “risky” patients (>35 years old, previously miscarried, etc.), pre-implantation genetic testing (PGT) may be indicated to aid in proper embryo selection. PGT provides information about the genetic makeup of the embryo, but not objective quality. Additionally, up to 5% of embryos undergoing biopsies for PGT are destroyed.

In 2017, the average age of first-time mothers in the U.S. was 26.8 years old, and this number has been steadily increasing since the 1970s. As this number likely will continue to increase, demand for assisted reproductive technologies and IVF in particular will grow. Thus, there remains a need for identifying high-quality oocytes for ART procedures and methods for improving the quality of oocytes.

### BRIEF SUMMARY OF THE INVENTION

The disclosure provides a method for selecting an oocyte for an assisted reproductive technology (ART) procedure, which method comprises: (a) assessing expression of the miRNAs miR-20b, miR-199, and miR-363 in a cumulus cell surrounding an oocyte isolated from a subject, (b) selecting an oocyte for an ART procedure if the cumulus cell expresses miR-20b, miR-199, and miR-363, and (c) performing an ART procedure using the selected oocyte.

The disclosure also provides method of improving the quality of an oocyte for an assisted reproductive technology (ART) procedure, which method comprises: (a) providing an oocyte isolated from a subject in which endogenous miR-20b, miR-199, and miR-363 expression is reduced or absent; and (b) culturing the oocyte in the presence of exogenous miR-20b, miR-199, and miR-363, whereby the quality of the oocyte is improved upon culturing in the presence of exogenous miR-20b, miR-199, and miR-363.

The disclosure further provides method for performing an assisted reproductive technology (ART) procedure on a female subject, which method comprises: (a) isolating one or more oocytes from a female subject; (b) assessing expression of the miRNAs miR-20b, miR-199, and miR-363 in a cumulus cell surrounding an isolated oocyte, and (c) (i) performing an ART procedure on the female subject with an oocyte whose cumulus cell expresses miR-20b, miR-199, and miR-363 or (ii) culturing an oocyte in which cumulus cell expression of miR-20b, miR-199, and miR-363 is reduced or absent in the presence of exogenous miRNAs miR-20b, miR-199, and miR-363 then performing an ART procedure on the female subject with the cultured oocyte.

Certain aspects of the presently disclosed subject matter having been stated hereinabove, which are addressed in whole or in part by the presently disclosed subject matter, other aspects will become evident as the description proceeds when taken in connection with the accompanying Example as best described herein below.

### DETAILED DESCRIPTION OF THE INVENTION

The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

The present disclosure is predicated, at least in part, on the discovery that the microRNAs (miRNAs) miR-199, miR-20b, and miR-363 are significantly downregulated in oocytes from aged women (older than 35 years), and can serve as biomarkers for higher quality, younger oocytes for use in assisted reproductive technologies (ARTs).

### Definitions

To facilitate an understanding of the present technology, a number of terms and phrases are defined below. Additional definitions are set forth throughout the detailed description.

MicroRNAs (miRNAs), also known as “mature miRNA” are small (approximately 18-24 nucleotides in length), non-coding RNA molecules present in the genomes of plants and animals. In certain instances, highly conserved, endogenously expressed miRNAs regulate the expression of genes by binding to the 3′-untranslated regions (3′-UTR) of specific mRNAs. More than 1000 different miRNAs have been identified in plants and animals. Certain mature miRNAs appear to originate from long endogenous primary miRNA transcripts (also known as pri-miRNAs, pri-mirs, pri-miRs or pri-pre-miRNAs) that are often hundreds of nucleotides in length (Lee, et al., EMBO J., 21(17): 4663-4670 (2002)).

The term “cumulus cells,” as used herein, refers to a group of closely associated granulosa cells that surround an oocyte and participate in the processes of oocyte maturation and fertilization. Cumulus cell function is dependent on gap junctions that form between cumulus cells and oocytes.

The term “assisted reproductive technology (ART)” refers to all fertility treatments in which both eggs and embryos are handled. In general, ART procedures involve surgically removing eggs from a woman's ovaries, combining them with sperm in a laboratory, and returning them to the woman's body or donating them to another woman. ART procedures do not include treatments in which only sperm are handled (e.g., intrauterine insemination) or procedures in which a woman takes medication only to stimulate egg production without the intention of having eggs retrieved. Examples of ART procedures include in vitro fertilization-embryo transfer (IVF-ET), gamete intrafallopian transfer (GIFT), zygote intrafallopian transfer (ZIFT), frozen embryo transfer (FET), and oocyte cryopreservation.

The term “oocyte,” as used herein, refers to an immature female gamete, ovum, or egg cell. The term “competent oocyte,” as used herein, refers to a female gamete or egg that, when fertilized, produces a viable embryo with a high implantation rate leading to pregnancy.

The term “embryo” refers to a fertilized oocyte or zygote.

As used herein, the term “modify” means changing a material from its initial state. For example, “modifying culture media” may include adding one or more miRNAs to the culture media or a component thereof or depleting one or more miRNAs from the culture media or a component thereof. Components of culture media may include a liquid growth media (e.g., classical media published by Dulbecco, Eagle, Ham, Moore, Morgan, and others) and a protein supplement. Accordingly, “modifying culture media” may include adding one or more miRNAs to a protein supplement or depleting one or more miRNAs from a protein supplement, wherein the protein supplement is added to a liquid growth media to form a culture media.

The terms “individual,” “host,” “subject,” and “patient” are used interchangeably herein, and generally refer to a female mammal, including, but not limited to, female primates, including simians and humans. Preferably, the subject is a human female.

The term “exogenous,” “non-native,” and “heterologous,” when describing a nucleic acid sequence (e.g., an miRNA) is any nucleic acid sequence (e.g., DNA, miRNA, RNA, or cDNA sequence) that is not a naturally occurring nucleic acid sequence of a cell, or is a naturally occurring nucleic acid that is located in a non-naturally occurring position in the cellular genome. In contrast, an “endogenous,” “native,” or “naturally occurring” nucleic acid sequence is any nucleic acid sequence (e.g., DNA, miRNA, RNA, or cDNA sequence) that is a naturally occurring nucleic acid sequence of a cell in a naturally occurring position.

### Selection of High-Quality Oocytes for ART

The disclosure provides a method for selecting an oocyte for an assisted reproductive technology (ART) procedure. The method comprises (a) assessing expression of the miRNAs miR-20b, miR-199, and miR-363 in a cumulus cell surrounding an oocyte isolated from a subject, (b) selecting an oocyte for an ART procedure if the cumulus cell expresses miR-20b, miR-199, and miR-363, and (c) performing an ART procedure using the selected oocyte. It will be appreciated that legal and ethical considerations make direct detection of biomarkers in oocytes or embryos difficult to implement. Thus, cumulus and mural granulosa cells reflect the characteristics of the oocyte, providing a noninvasive means to assess oocyte quality (see, e.g., McKenzie et al., Hum Reprod, 19: 2869-74 (2004); van Montfoort et al., Mol Hum Reprod, 14: 157-68 (2008); Zhang et al., Fertil Steril, 83(Suppl 1): 1169-79 (2005); Assou et al., Molecular Human Reproduction, 14(12): 711-719 (2008); and Hamel et al., Hum Reprod, 23: 1118-27 (2008)).

Methods for retrieving oocytes are well known in the art and may be used in connection with the methods disclosed herein. For ART procedures in human females, oocyte retrieval typically is performed via transvaginal oocyte retrieval (TVOR) (also referred to as oocyte retrieval (OCR) or transvaginal ovum retrieval), in which oocytes are retrieved using conscious sedation via a transvaginal approach under ultrasound guidance with low-pressure aspiration (see, e.g., Healy et al., Semin Reprod Med., 33(2): 83-91 (2015) and Dellenbach et al., Lancet, 1(8392): 146 (1984)). Methods for isolating and culturing cumulus cells are known in the art and are described in, e.g., Li et al., Biol Reprod., 63(3): 839-45 (2000); and Chilvers et al., J Assist Reprod Genet., 29(6): 547-5 (2012); Assou et al., Stem Cells Dev., 24(19): 2317-2327 (2015)). Oocytes and cumulus cells desirably are retrieved and isolated from a human female, and preferably a human female of reproductive age. In some embodiments, the human female is at least 35 years old.

Functional analyses of miRNAs have revealed that these small non-coding RNAs contribute to different physiological processes in animals, including developmental timing, organogenesis, differentiation, patterning, embryogenesis, growth control, and programmed cell death. For example, miRNAs play a role in stem cell differentiation, neurogenesis, angiogenesis, hematopoiesis, and exocytosis (see, e.g., Alvarez-Garcia and Miska, Development, 132: 4653-4662 (2005)). More recently, miRNAs have been identified that are involved in embryo viability and chromosomal makeup (see, e.g., U.S. Patent Application Publication No. 2014/0296099 A1 and U.S. Pat. No. 10,059,994). The inventors of the present disclosure have surprisingly found that expression of the miRNAs miR-20b, miR-199, and miR-363 are associated with overall health and quality of human oocytes. Nucleotide sequences of miR-20b (also referred to as “hsa-miR-20b”), miR-199 (also referred to as “has-miR-199”), and miR-363 (also referred to as “has-miR-363”) are publicly available from the National Center for Biotechnology Information (NCBI) as indicated in Table 1.

TABLE 1

| miRNA | NCBI Accession No. |
|---|---|
| miR-20b | NR_029950.1 |
| miR-199 | NR_029586.1 |
| miR-363 | NR_029852.1 |

Expression of the miRNAs miR-20b, miR-199, and miR-363 may be assessed using any suitable method for detecting and measuring gene expression known in the art. In some embodiments, detecting expression of miRNA may include detecting nucleic acid comprising miRNA, pre-miRNA, or pri-miRNA. In other embodiments, microRNA expression may be assessed via detecting nucleic acid comprising miRNA, pre-miRNA, or pri-miRNA in an extracellular sample (e.g., in culture media in which an oocyte or cumulus cell is growing) or in an intracellular sample (e.g., an intracellular sample of an oocyte or cumulus cells). Exemplary methods for assessing expression of miRNAs include, but are not limited to, RT-PCR, quantitative or real-time RT-PCR (qRT-PCR), microarray analysis, RNA sequencing, in situ hybridization, or Northern blot. In certain embodiments, miRNA expression is assessed using RT-PCR. RT-PCR involves converting miRNA to cDNA via reverse transcription and amplifying the cDNA via polymerase chain reaction. RNA linkers may be ligated to the miRNA prior to converting the miRNA to cDNA and/or cDNA linkers may be ligated to the cDNA prior to amplifying the cDNA. Multiple miRNAs may be detected in the described methods (e.g., at least 2, 3, 4, 5, 6, 7, 8, 9, 10, 11, 12, 13, 14, 15, 16, 17, 18, 19, or 20 miRNAs) and microarrays comprising probes for multiple miRNAs may be utilized to detect multiple miRNAs. Methods for detecting and assessing miRNA expression are described in detail in, e.g., Baker, M., Nat Methods, 7(9): 687-92 (2010); de Planell-Saguer M., Rodicio M.C., Clinical Biochemistry, 46: 869-878 (2013); Kappel, A. and Keller, A., Clin. Chem. Lab Med., 55(5): 636-647 (2017); and Tian et al., Org. Biomol. Chem., 13: 2226-2238 (2015).

If a cumulus cell is determined to express miR-20b, miR-199, and miR-363, the method further comprises selecting the oocyte from which the cumulus cell is obtained for use in an ART procedure. If a cumulus cell does not express, downregulates, or expresses miR-20b, miR-199, and miR-363 at low levels, then the above described method may be performed on a cumulus cell isolated from a different oocyte. Expression of miR-20b is downregulated if the miR-20b expression detected in the cumulus cell is reduced by at least about 35% (e.g., 40%, 45%, 50%, 55%, 60%, 65%, 70%, 75%, 80%, 85%, 90%, 95%, 99% or more) as compared to a reference level or control small nucleolar RNA (snoRNA). Expression of miR-199 is downregulated if the miR-199 expression detected in the cumulus cell is reduced by at least about 58% (e.g., about 60%, 65%, 70%, 75%, 80%, 85%, 90%, 95%, 99% or more) as compared to a reference level or control snoRNA. Expression of miR-363 is downregulated if the miR-363 expression detected in the cumulus cell is reduced by at least about 47% (e.g., 50%, 55%, 60%, 65%, 70%, 75%, 80%, 85%, 90%, 95%, 99% or more) as compared to a reference level or control snoRNA.

Any suitable ART procedure may be performed using the selected oocyte. As discussed above, exemplary ART procedures include, but are not limited to, in vitro fertilization-embryo transfer (IVF-ET), gamete intrafallopian transfer (GIFT), zygote intrafallopian transfer (ZIFT), oocyte cryopreservation, and frozen embryo transfer (FET). The term “in vitro fertilization” or “IVF” refers to a process by which oocytes are fertilized by sperm outside of the body, in vitro, to produce an embryo. The embryo is then introduced into the female's uterus, where it may implant and lead to a successful pregnancy. GIFT involves removing a eggs from a female, mixing them with sperm, and immediately placing them back into the female's fallopian tube. ZIFT combines IVG and GIFT, by stimulating and collecting eggs using IVF methods. The eggs are then mixed with sperm in a laboratory, and fertilized eggs (embryos) are then laparoscopically returned to the fallopian tubes where they are carried into the uterus. Oocyte cryopreservation is a process in which a woman's oocytes are extracted, frozen, and stored as a method to preserve reproductive potential in women of reproductive age. FET is a process in which previously cryopreserved embryo(s) are thawed and transferred into the patient's uterus at the appropriate time in her menstrual cycle. Assisted reproductive technologies are further described in, e.g., Gardener et al. (eds.), Textbook of Assisted Reproductive Techniques: Two Volume Set, 5th Edition, CRC Press (2018).

### Method of Improving Oocyte Quality for ART

The disclosure also provides a method of improving the quality of an oocyte for an assisted reproductive technology (ART) procedure, (a) providing an oocyte isolated from a subject in which endogenous miR-20b, miR-199, and miR-363 expression is reduced or absent; and (b) culturing the oocyte in the presence of exogenous miR-20b, miR-199, and miR-363, whereby the quality of the oocyte is improved upon culturing in the presence of exogenous miR-20b, miR-199, and miR-363. Descriptions of oocytes, ART procedures, and components thereof set forth above in connection with the method of selecting an oocyte for an ART procedure as described above also apply to those same aspects of the aforementioned method of improving oocyte quality.

One of ordinary skill in the art will appreciate that oocyte quality has a direct impact on the fertilization and developmental competence of oocytes. Oocyte quality typically is evaluated using morphological, cellular, and molecular criteria. Morphological measures of oocyte quality generally focus on the structure of the oocyte: cumulus complex, oocyte cytoplasm, polar body, perivitelline space, zona pellucida, and meiotic spindle. Cellular and molecular markers of oocyte quality include, but are not limited to, mitochondrial status and glucose-6-phosphate dehydrogenase 1 activity, apoptosis of follicular cells, and levels of the transforming growth factor-0 superfamily in follicular fluid or serum (see, e.g., Wang, Q. and Sun, Q. Y., Reprod Fertil Dev., 19(1): 1-12 (2007); and Lasienëet al., Medicina (Kaunas), 45(7): 509-515 (2009)). As discussed herein, molecular markers of oocyte quality also include expression of certain miRNAs, such as miR-20b, miR-199, and miR-363.

Desirably, the method comprises providing an oocyte isolated from a subject in which endogenous miR-20b, miR-199, and miR-363 expression is reduced or absent, and culturing the oocyte in the presence of exogenous miR-20b, miR-199, and miR-363. Expression of miR-20b, miR-199, and miR-363 in an oocyte retrieved from a female subject may be assessed by performing any of the methods described herein on one or more cumulus cells isolated from the oocyte. Expression of the miRNAs is “absent” if miR-20b, miR-199, and miR-363 are not present in levels detectable by the particular assay used. As discussed above, expression of the miRNAs is “reduced” or “downregulated” if miR-20b, miR-199, and miR-363 are detectable but reduced by at least about 35%, 58%, and 47%, respectively, as compared a reference level or control small nucleolar RNA (snoRNA).

In some embodiments, the culture media in which the oocytes are maintained and grown may be supplemented with exogenous miR-20b, miR-199, and miR-363 sequences. In other embodiments, the exogenous miR-20b, miR-199, and miR-363 are present in extracellular vesicles. The term “extracellular vesicles (EVs),” as used herein, refers to small (30-1000 nm) vesicles released by cells either by outward budding from the plasma membrane or via exocytosis (Théry et al., Nat Rev Immunol., 2(8): 569-79 (2002)). Upon shedding, EVs may fuse with recipient cells directly or after endocytosis followed by the release of contents, or interact with target cells by receptor-ligand interactions. Since EVs carry proteins, mRNAs, and miRNAs from their parental cells, they represent an important component of paracrine signaling and cell-to-cell communication (Valadi et al., Nat Cell Biol., 9(6): 654-9 (2007)). Thus, it is believed that extracellular vesicles containing miR-20b, miR-199, and miR-363 may ultimately increase the endogenous expression of miR-20b, miR-199, and miR-363 in an oocyte from a woman of advanced age (35 years or older) or in an oocyte of lower quality from a young woman (less than 35 years) when introduced into the culture medium wherein oocytes are matured. Because microRNAs affect regulatory processes in cells, the increased presence of miR-20b, miR-199, and miR-363 in cultured oocytes will enhance endogenous expression of these miRNAs and associated processes. The extracellular vesicles may be obtained by any suitable method or procedure. In some embodiments, EVs containing miR-20b, miR-199, and miR-363 can be obtained by generating induced pluripotent stem cells (iPSCs) from blood cells isolated from a young female with presumably high quality oocytes, and collecting EVs from the culture media of the iPSC cells. Methods for isolating EVs from iPSCs are known in the art and described in, e.g., Chen et al., Methods Mol Biol., 1660: 389-394 (2017); Adamiak et al., Circ Res., 122(2): 296-309 (2018); and Liu et al., Stem Cells, 37(6): 779-790 (2019)).

In other embodiments, improving the quality of an oocyte may comprise modifying an oocyte isolated from a subject in which miR-20b, miR-199, and miR-363 expression is reduced or absent. An oocyte that does not express miRNAs miR-20b, miR-199, and miR-363, or expresses miRNAs miR-20b, miR-199, and miR-363 at low levels, may be modified to express miRNAs miR-20b, miR-199, and miR-363 using routine methods for introducing exogenous nucleic acids into cells. For example, miRNAs may be introduced into a cell by “transfection,” “transformation,” or “transduction.” “Transfection,” “transformation,” or “transduction,” as used herein, refer to the introduction of one or more exogenous polynucleotides into a cell by using physical or chemical methods. Many transfection techniques are known in the art and include, for example, DEAE-dextran; electroporation; cationic liposome-mediated transfection; tungsten particle-facilitated microparticle bombardment; and the use of viral, phage, or plasmid vectors (see, e.g., Murray E. J. (ed.), Methods in Molecular Biology, Vol. 7, Gene Transfer and Expression Protocols, Humana Press (1991); Brash et al., Mol. Cell Biol., 7: 2031-2034 (1987); and Johnston, Nature, 346: 776-777 (1990)). Commercially-available RNA transfection systems may also be used in the methods described herein.

The disclosure further provides a method for performing an assisted reproductive technology (ART) procedure on a female subject, which method comprises: (a) isolating one or more oocytes from a female subject; (b) assessing expression of the miRNAs miR-20b, miR-199, and miR-363 in a cumulus cell surrounding an isolated oocyte, and (c) (i) performing an ART procedure on the female subject with an oocyte whose cumulus cell expresses miR-20b, miR-199, and miR-363 or (ii) culturing an oocyte in which cumulus cell expression of miR-20b, miR-199, and miR-363 is reduced or absent in the presence of exogenous miRNAs miR-20b, miR-199, and miR-363 then performing an ART procedure on the female subject with the cultured oocyte. Descriptions of oocyte and cumulus cell isolation, assessment of miRNA expression, oocyte culture, ART procedures, and components thereof as described above also apply to those same aspects of the method for performing an ART procedure on a female subject. The female subject desirably is a human female of reproductive age. In some embodiments, the human female is at least 35 years old.

### Kits

Reagents for detecting each of the miRNAs miR-20b, miR-199, and miR-363 can be provided in a kit, i.e., a packaged combination of reagents in predetermined amounts with instructions for detecting the miRNAs. Reagents include, for example, oligonucleotides that hybridize specifically to one or more of miR-20b, miR-199, and miR-363 and detect the miRNA based on the specific hybridization. For example, the oligonucleotide reagents may include one or more primers for performing any or all steps of RT-PCR performed on miRNA as contemplated herein (i.e., a primer for performing reverse transcription (RT) of an miRNA to obtain reverse transcribed miRNA and/or one or a pair of primers for performing polymerase chain reaction (PCR) of the reverse transcribed miRNA to obtain an amplified product). The reverse transcribed miRNA or amplified product then may be detected by methods known in the art and described herein. Oligonucleotide reagents may include probes for detecting an miRNA and/or any product of RT-PCR performed on miRNA (e.g., a probe for detecting a reverse transcribed miRNA, or a probe for detecting an amplified product of the reverse transcribed miRNA). Primers and probes as contemplated herein may include a detectable label.

The instructions can be in paper form or computer-readable form, such as a disk, CD, DVD, etc. Alternatively or additionally, the kit can comprise a calibrator or control, and/or at least one container (e.g., tube, microtiter plates, or strips) for conducting an assay, enzymes for performing RT-PCR (e.g., a reverse transcriptase or a DNA polymerase, such as a thermostable polymerase) and/or buffers. Ideally, the kit comprises all components, i.e., reagents, standards, buffers, diluents, etc., which are necessary to assess miRNA expression. Other additives may be included in the kit, such as stabilizers, buffers, and the like. The relative amounts of the various reagents can be varied to provide for concentrations in solution of the reagents which substantially optimize the sensitivity of the assay. The reagents may be provided as dry powders (typically lyophilized), including excipients which on dissolution will provide a reagent solution having the appropriate concentration.

The following Example has been included to provide guidance to one of ordinary skill in the art for practicing representative embodiments of the presently disclosed subject matter. In light of the present disclosure and the general level of skill in the art, those of skill can appreciate that the following Example is intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### EXAMPLE

This example demonstrates lower expression of microRNAs miR-199, miR-20b, and miR-363 in aging women.

Experiments were conducted to determine whether miRNAs were differentially expressed in aging women. Significant changes were observed in up to 20 miRNAs (miR-486-5p, miR-486-3p, miR-1246, miR-363-3p, miR-451a, miR-20b-5p, miR-142-5p, miR-142-3p, miR-145-5p, miR-663a-5p, miR-625-3p, miR-625-5p, miR-199a-5p, miR-199a-3p, and miR-199b-3p) when compared between women less than 35 years of age and above 40 years of age. Furthermore, high density qPCR was performed to validate the changes in miRNA expression with additional samples from the same two groups (i.e., less than 35 years of age and above 40 years of age). The miRNAs miR-199, miR-20b, and miR-363 were significantly downregulated in aged women (above 40 years of age).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1-10. (canceled)

11. A method for selecting an oocyte for an assisted reproductive technology (ART) procedure, which method comprises:
(a) assessing expression of the miRNAs miR-20b, miR-199, and miR-363 in a cumulus cell surrounding an oocyte isolated from a subject;
(b) selecting an oocyte for an ART procedure if the isolated cumulus cell expresses miR-20b, miR-199, and miR-363; and
(c) performing an ART procedure using the selected oocyte.

12. The method of claim 11, wherein assessing expression of the miRNAs miR-20b, miR-199, and miR-363 comprises qRT-PCR, microarray analysis, RNA sequencing, In situ hybridization, or Northern blot.

13. The method of claim 11, wherein the assisted reproductive technology procedure is in vitro fertilization (IVF).

14. The method of claim 11, wherein the assisted reproductive technology procedure is oocyte cryopreservation.

15. The method of claim 11, wherein the subject is a female at least 35 years old.

16. A method of improving the quality of an oocyte for an assisted reproductive technology (ART) procedure, which method comprises:
(a) providing an oocyte isolated from a subject in which endogenous miR-20b, miR-199, and miR-363 expression is reduced or absent; and
(b) culturing the oocyte in the presence of exogenous miR-20b, miR-199, and miR-363, whereby the quality of the oocyte is improved upon culturing the oocyte in the presence of exogenous miR-20b, miR-199, and miR-363.

17. The method of claim 16, wherein the exogenous miR-20b, miR-199, and miR-363 are provided in a cell culture medium.

18. The method of claim 16, wherein the exogenous miR-20b, miR-199, and miR-363 are present in extracellular vesicles.

19. The method of claim 16, wherein the assisted reproductive technology procedure is in vitro fertilization (IVF).

20. The method of claim 16, wherein the assisted reproductive technology procedure is oocyte cryopreservation.

21. The method of claim 16, wherein the subject is a female at least 35 years old.

22. A method for performing an assisted reproductive technology (ART) procedure on a female subject, which method comprises:
(a) isolating one or more oocytes from a female subject;
(b) assessing expression of the miRNAs miR-20b, miR-199, and miR-363 in a cumulus cell surrounding an isolated oocyte, and
(c) (i) performing an ART procedure on the female subject with an oocyte whose cumulus cell expresses miR-20b, miR-199, and miR-363; or
(ii) culturing an oocyte in which cumulus cell expression of miR-20b, miR-199, and miR-363 is reduced or absent in the presence of exogenous miRNAs miR-20b, miR-199, and miR-363; and performing an ART procedure on the female subject with the cultured oocyte.

23. The method of claim 22, wherein assessing expression of the miRNAs miR-20b, miR-199, and miR-363 comprises qRT-PCR, microarray analysis, RNA sequencing, In situ hybridization, or Northern blot.

24. The method of claim 22, wherein the assisted reproductive technology procedure is in vitro fertilization (IVF).

25. The method of claim 22, wherein the assisted reproductive technology procedure is oocyte cryopreservation.

26. The method of claim 22, wherein the subject is a female at least 35 years old.
